(19) United States
(12) Patent Application Publication
(10) Pub. No.: US 2012/0289523 A1
(43) Pub. Date: Nov. 15, 2012

(54) PYRROLO [3,2-E] [1,2,4] TRIAZOLO [1,5-A] PYRIMIDINES DERIVATIVES AS INHIBITORS OF MICROGLIA ACTIVATION

(75) Inventor: David Scopes, Cambridgeshire (GB)

(73) Assignees: BTG INTERNATIONAL LIMITED, Greater London (GB); SENEXIS LIMITED, Cambridgeshire (GB)

(21) Appl. No.: 13/501,014

(22) PCT Filed: Oct. 7, 2010
(86) PCT No.: PCT/EP10/65001

§ 371 Date: Jul. 31, 2012

(30) Foreign Application Priority Data
Oct. 9, 2009 (GB) 0917775.9

(51) Int. Cl. A61K 31/519; A61P 25/28; A61P 25/16; C07D 487/14

(52) U.S. Cl. 514/267; 544/251

(57) ABSTRACT

The invention relates to novel compounds useful in the treatment and prophylaxis of disease. Compounds of the formula (I) wherein X is halogen, independently selected form chlorine and fluorine and n is 0, 1 or 2, and their pharmaceutically acceptable salts are useful in the treatment and prophylaxis of diseases caused by activation of microglia, particularly Alzheimer's disease.

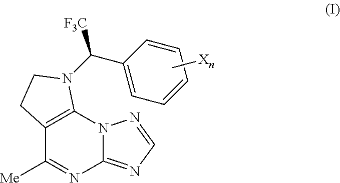

This invention relates to novel compounds useful in the treatment and prophylaxis of disease. Particularly the current invention provides compounds useful in the treatment and prophylaxis of diseases caused by activation of microglia, particularly where the activation is caused by amyloid proteins such as β amyloid.

In the development of pharmaceutically active compounds, the provision of compounds with improved activity (e.g. at the target site, or in model systems) is important. However, it is also important that active compounds have useful pharmacokinetic, pharmacodynamic and toxicological properties. Consequently, in some cases, high activity may be balanced against other properties such as bioavailability, in vivo half life, cell permeability, appropriate resistance to metabolism and a low probability of adverse interactions with other drugs. Adverse interactions can occur, for example, when one drug retards the metabolism of another, for example through interaction with metabolic enzymes. Thus low affinity for cytochrome P450s, especially those involved in drug metabolism, is also important. This is especially so if the subject to be treated suffers additional symptoms that are treated by further drugs.

EP1433480 discloses the use of certain pyrimidine derivatives for the treatment of central nervous system diseases. Uryu et al (2002) Brain Research, 946(2), 298-306 and Uryu et al (2003) Biochem. Biophys. Res. Com., 303(1), 302-305, both discuss RS-1178, a compound recited in EP1433480.

U.S. Pat. No. 4,007,189 describes pyrrolotriazolopyrimidine derivatives which are said to be useful as antihypertensive agents. JP 52116497 describes triazolopyrimidines said to be useful as vasodilators and antihypertensives, Y. Sato et al., J. Med. Chem. (1980), 23, 927-937 describes 1,2,4-triazolo[1,5-a]pyrimidines fused to heterocyclic systems, which are said to be useful as vasodilators. EP347252 describes triazolo- and pyrazolopyrrolopyrimidines useful in the treatment of cachexia.

The current invention provides specific, novel compounds of the formula (I), that are not disclosed in EP1433480, that are potent inhibitors of the activation of macrophages, that are useful as pharmaceutical actives in the treatment of disease and which provide improved properties compared to compounds of the prior art.

Thus a first aspect of the invention provides a compound of the formula (I) or a pharmaceutically acceptable salt thereof:

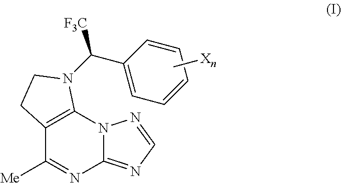

wherein
X is halogen, independently selected form chlorine and fluorine,
n is 0, 1 or 2
Preferably X is fluorine. Halogen X, when present, is preferably in the 3-position, the 4-position, or is in the 3- and 4-position; preferably, where present, it is in the 4-position, or is in the 3- and 4-position; and is most preferably in the 4-position.
Preferably n is either 0 or 1 and particularly 1.

preferred compounds of the invention are

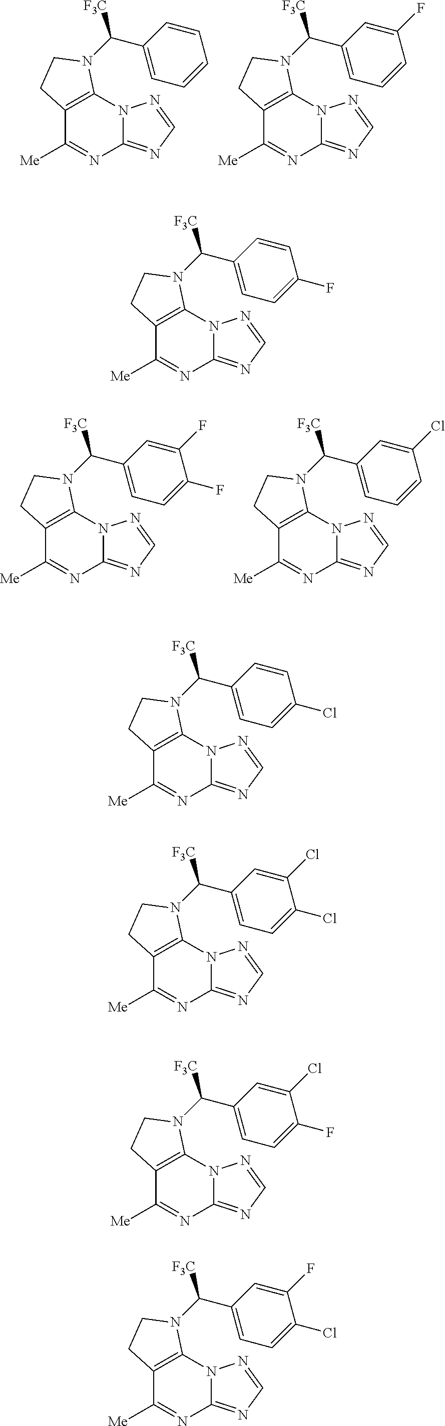

Particularly preferred are compounds 1, 2 and 3 below and most preferably compounds 1 and 2.

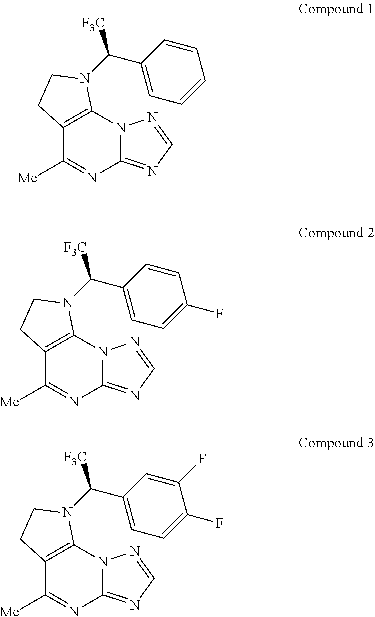

Preferred pharmaceutically acceptable salts include those formed with strong acids such as hydrochloric acid, sulfuric acid, phosphoric acid, methanesulfonic acid, benzene sulfonic acid and particularly hydrochloric acid and methanesulfonic acid.

A second embodiment of the invention provides the use of a compound of the formula (I) or a pharmaceutically acceptable salt thereof, in therapy.

Compounds of the formula (I) are potent inhibitors of the activation of macrophages in vitro, via a pathway that differs from that by which lipopolysaccharides and zymosan act (EP1433480). This system is used as a model for microglial activation (Uryu et al (2002) Brain Research, 946(2), 298-306). The compounds of the invention are therefore useful in conditions in which microglial activation plays a role. Microglial activation has been proposed in a number of mammalian neurodegenerative conditions, particularly in Alzheimer's disease, Parkinson's disease (e.g. Teisman and Schulz 2004), Huntington's chorea (e.g. Bonifati and Kishore 2006) and Pick's disease (e.g. Schofield et al 2003). Compounds of the formula (I) have particularly been demonstrated to be inhibitors of macrophage activation by amyloid protein and so are particularly useful in conditions in which activation is induced by amyloid proteins, particularly in Parkinson's disease and Alzheimer's disease.

Compounds of the invention may be used without further components to the composition, that is to say that the composition consists essentially of the compound of the invention, but will generally be used as a pharmaceutically acceptable composition, which optionally comprises one or more pharmaceutically acceptable carriers or diluents. The compounds will generally be provided in a composition that is sterile and pyrogen free.

Preparations suitable for any of the commonly used routes of administration such as oral, rectal, nasal, topical or parenteral may be prepared by methods well known in the art of pharmacy. These may take the form of solutions, suspensions, tablets, pills, capsules, powders, sustained release formulations and the like.

Suitable doses of the compounds of the invention will be in the range 0.1 mg of compound per kg body weight to 100 mg/kg, preferably 1 mg/kg to 100 mg/kg and more preferably 1 mg/kg to 10 mg/kg.

A third embodiment of the invention provides a pharmaceutical composition comprising a compound of the formula (I) or a pharmaceutically acceptable salt thereof, preferably in combination with a pharmaceutically acceptable carrier or diluent.

In addition, the compounds of the invention may be administered with one or more additional therapeutic compounds. For example, one or more anti-inflammatory compounds (eg NSAIDS), which have been shown to slow the onset of neurodegenerative diseases; one or more compounds suitable for the treatment of Alzheimer's disease (eg beta-amyloid aggregation inhibitors, gamma-secretase inhibitors, gamma-secretase modulators or beta-secretase inhibitors); or compounds for the treatment of Parkinson's disease. Thus, the pharmaceutical formulations of the invention can additionally comprise such compounds. However, it is of course possible to administer such compounds separately, either at the same time as a compound of the invention or sequentially.

Thus, in a fourth aspect, the present invention provides a composition comprising a compound of the invention, together with one or more additional therapeutic compounds for simultaneous, sequential or separate use. The one or more additional compounds can be chosen from the examples discussed above.

A fifth aspect of the invention provides a method of treatment of diseases involving the activation of microglia (particularly where microglia are activated by amyloid protein), comprising administering to a patient in need thereof, a therapeutically effective amount of a compound of the formula (I) or a pharmaceutically acceptable salt thereof.

A sixth aspect of the invention provides the use of a compound of the formula (I) or a pharmaceutically acceptable salt thereof in the manufacture of a medicament for the treatment of diseases involving the activation of microglia (particularly where microglia are activated by amyloid protein).

A seventh aspect of the invention provides a compound of the formula (I) or a pharmaceutically acceptable salt thereof for the treatment of diseases involving the activation of microglia (particularly where microglia are activated by amyloid protein).

The current invention will now be described with the help of the following examples, schemes and figures. Further embodiments within the scope of the invention will become apparent to the skilled worker in the light of these.

### General Synthetic Routes

Synthesis of compounds in which X and Y are independently H, Cl or F is readily achieved via the route shown in Scheme 1 starting from the commercially available trifluoromethyl ketones (7). Reaction of triflates (9) of the alcohols (8) with the secondary amine (10) provides the racemic analogues.

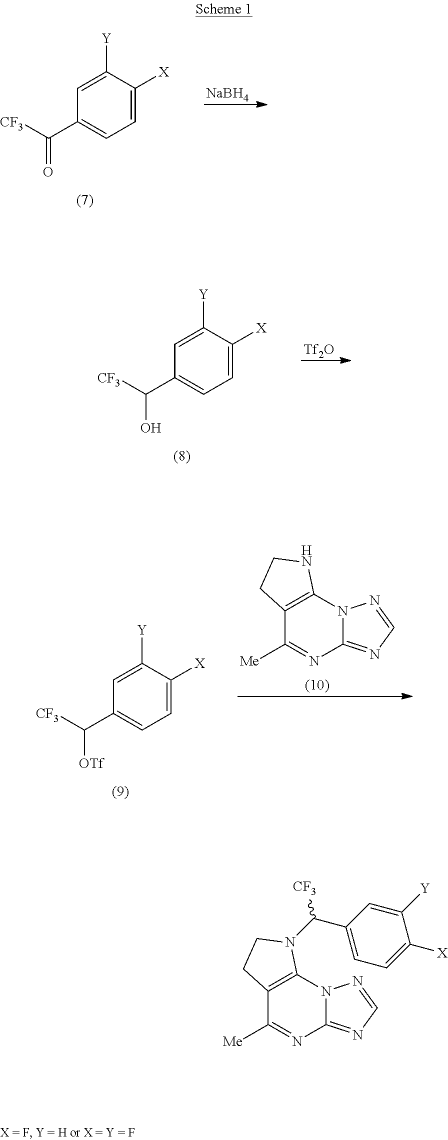

Chiral syntheses are accomplished via chiral reduction of the appropriate trifluoromethyl ketones, employing catecholborane with 1-(R)-methyl-CBS-oxazaborolidine or 1-(S)-methyl-CBS-oxazaborolidine catalyst (Schemes 2a and 2b, respectively). Final chiral purification is achieved after the final step using a CHIRALPAK® AD-H HPLC column and gives materials with high enantiomeric excess (ee).

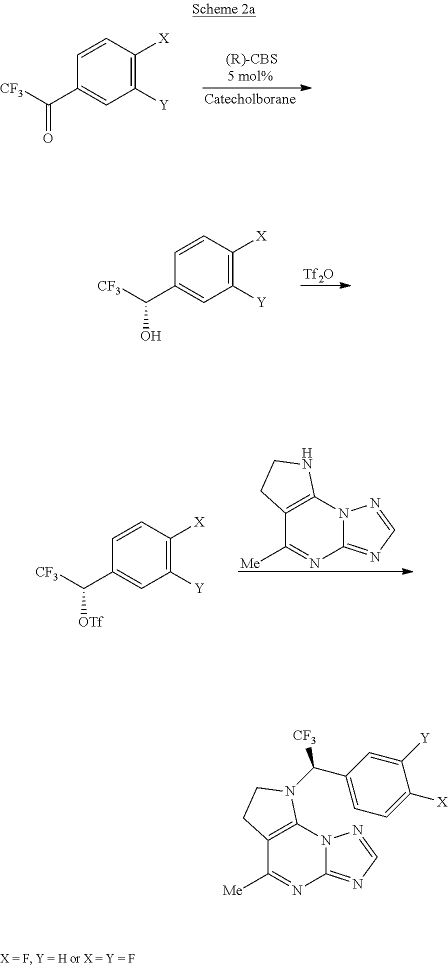

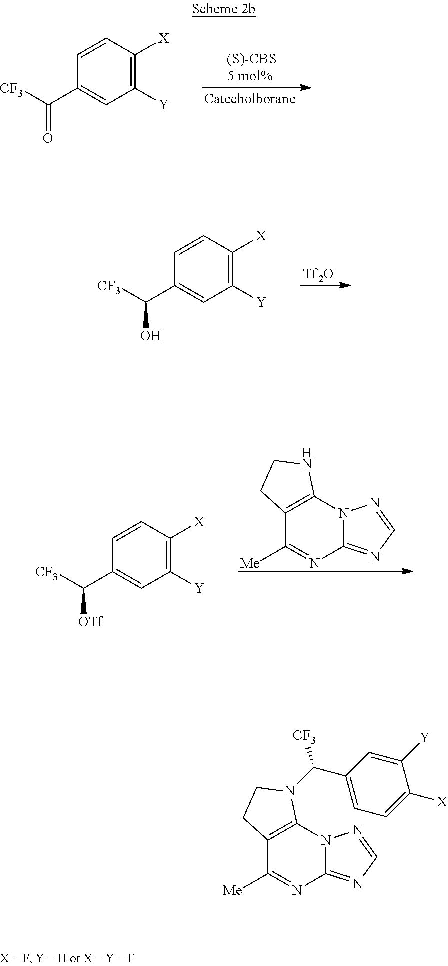

### SYNTHETIC EXAMPLES

### Example 1

### (R)-5-Methyl-8-[2,2,2-trifluoro-1-phenylethyl]-7,8-dihydro-6H-pyrrolo[3,2e][1,2,4]-triazolo[1,5-a]pyrimidine [Compound 1]

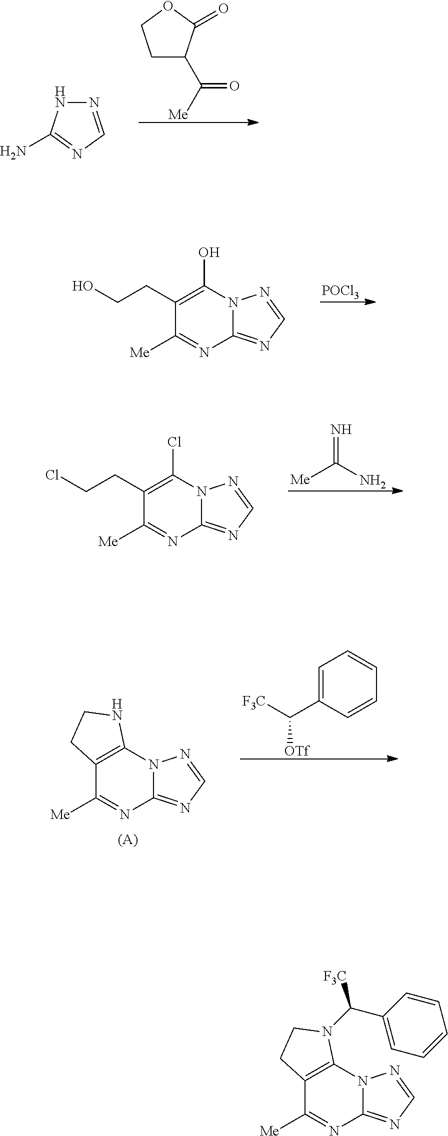

Intermediate (A) may be synthesised by the method of Y. Sato et al., J. Med. Chem. (1980) 23, 927-937.

### 6-(2-Hydroxyethyl)-5-methyl[1,2,4]triazolo[1,5-a]pyrimidin-7(4H)-one

A mixture of 3-aminotriazole (350 g, 4.1 mol) and α-acetyl-γ-butyrolactone (524.8 g, 4.1 mol) was stirred in IMS (2.8 L) and BF₃.Et₂O (85 mL, 0.6 mol) added over 15 min. After 3 days stirring at ambient temperature the solid was collected by filtration and dried on the filter.

¹H NMR (d6-DMSO) δ 13.6 (1H, br), 10.37 (1H, s), 8.40 (1H, s), 4.30 (2H, t), 2.88 (2H, t) and 2.50 (3H, s).

The solid was stirred in water (1.7 L) and triethylamine (422 mL, 4.1 mol) added, after which the solid dissolved. After stirring for 2 days at ambient temperature, acetic acid (1 eq. 90 mL) was added. The mixture was stirred for 1 h and the solid filtered, dried on the filter and under vacuum at 40° C. for 4 h to give the dihydroxy compound as a white solid, 430 g, 2.2 mol (54%).

¹H NMR (d6-DMSO) δ 8.16 (1H, s), 3.48 (2H, t), 2.62 (2H, t) and 2.36 (3H, s). ES⁺ 195 (100%), M+H⁺.

### 7-Chloro-6-(2-chloroethyl)-5-methyl[1,2,4]triazolo[1,5-a]pyrimidine

POCl₃ (130 mL) was added to 6-(2-hydroxyethyl)-5-methyl[1,2,4]triazolo[1,5-a]pyrimidin-7(4H)-one (111 g, 0.57 mol) in a single portion (generates an exotherm) and the mixture stirred and heated in 40° C. steps to 120° C. (at 70-80° C. all the solids dissolved). After 5 h heating was stopped and the mixture allowed to cool overnight. Some residual POCl₃ was removed under vacuum and the residue added to well-stirred water (1 L) over 40 min. The temperature rose upon addition and ice was added periodically to keep the temperature below 25° C., care being taken to avoid the gum settling below the water. The mixture was cooled in an ice-bath, stirred and the pH adjusted to approximately 7 with aqueous ammonia solution and the solid collected. The solid was taken into dichloromethane (150 mL), the separated water removed, any solids removed by filtration and the organic solution dried over MgSO₄. After concentration, the crude material was purified by elution under vacuum through silica (eluent: 1.5-2% methanol/dichloromethane) to give the dichloro compound as a white solid (62 g, 0.27 mol).

¹H NMR (d6-DMSO) δ 8.66 (1H, s), 3.90 (2H, t), 3.33 (2H, t) and 2.75 (3H, s). ES⁺ 231 (100%), M+H⁺.

### 5-Methyl-7,8-dihydro-6H-pyrrolo[3,2-e][1,2,4]triazolo[1,5-a]pyrimidine (1)

A mixture of 7-chloro-6-(2-chloroethyl)-5-methyl[1,2,4]triazolo[1,5-a]pyrimidine (3.50 g, 0.015 mol), acetamidine hydrochloride (1.4 g, 0.015 mol) and sodium carbonate (4.8 g, 0.045 mol) in ethanol (75 mL) was heated at 90° C. for 8 h. The reaction was monitored by TLC using dichloromethane: methanol (8:2) as a mobile phase. The mixture was filtered and concentrated. The crude product was purified by column chromatography on silica gel, using dichloromethane:methanol (8:2) as eluent, to afford the 5-methyl-7,8-dihydro-6H-pyrrolo[3,2-e][1,2,4]triazolo[1,5-a]pyrimidine as yellow solid (1.3 g, 49%).

Mass: (ES+) 176 (M+H)⁺

### (S)-(+)-1,1,1-Trifluoro-2-hydroxy-2-phenylethane, triflate

Trifluoromethanesulfonic anhydride (0.30 mL, 1.8 mmol) was added dropwise to an ice-cooled solution of (S)-(+)-1,1,1-trifluoro-2-hydroxy-2-phenylethane (0.18 g, 1.0 mmol, ee 97%) and 2,6-lutidine (0.21 g, 2 mmol) in dichloromethane (2 mL). The mixture was stirred with cooling for 10 min, poured into diethyl ether and washed rapidly with aqueous hydrochloric acid (pH=5) and brine. The solution was dried (MgSO₄) and the solvent removed under reduced pressure without heating. The crude triflate was obtained as an unstable pink oil (0.58 g) which was used immediately in the next step.

### (R)-5-Methyl-8-[2,2,2-trifluoro-1-phenylethyl]-7,8-dihydro-6H-pyrrolo[3,2e][1,2,4]-triazolo[1,5-a]pyrimidine

5-Methyl-7,8-dihydro-6H-pyrrolo[3,2-e][1,2,4]triazolo[1,5-a]pyrimidine (75 mg, 0.43 mmol) was added to a stirred suspension of sodium hydride (60% dispersion in mineral oil, 17 mg, 0.43 mmol) in DMF (4 mL). After 30 min, the triflate (198 mg, 0.64 mmol) was added and the mixture was stirred at room temperature overnight. The mixture was diluted with ethyl acetate and washed with water and brine, dried (MgSO₄) and the solvent removed under reduced pressure. The crude product was purified by column chromatography on silica gel (ethyl acetate/methanol) to afford the title compound as a light brown solid (24 mg, 17%).

¹H NMR (CDCl₃) δ 2.44 (3 H, s), 3.01-3.12 (1 H, m), 3.16-3.24 (1 H, m), 3.64 (1 H, m), 4.01-4.08 (1 H, m), 7.38-7.46 (5 H, m), 7.62 (1 H, q) and 8.37 (1 H, s). LCMS (ES⁺): 334 (MH⁺, 100%).

### Example 2

### (R)-5-Methyl-8-[2,2,2-trifluoro-1-(4-fluorophenyl)ethyl]-7,8-dihydro-6H-pyrrolo[3,2e][1,2,4]triazolo[1,5-a]pyrimidine [Compound 2]

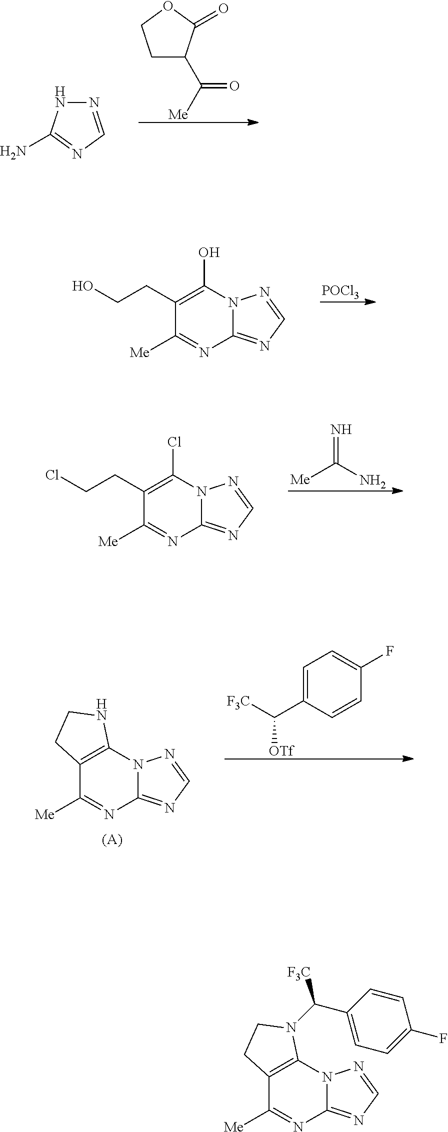

Intermediate (A) may be synthesised by the method of Y. Sato et al., J. Med. Chem. (1980) 23, 927-937.

### (S)-2,2,2-Trifluoro-1-(4-fluorophenyl)ethanol

To a 50 mL, 3-neck round-bottom flask, was added 2,2,2,4′-tetrafluoroacetophenone (0.5 g, 2.60 mmol) and anhydrous dichloromethane (10 mL). The flask was flushed with nitrogen and cooled to −78° C. To the resulting solution was added 2-(R)-methyl-CBS-oxazaborolidine (0.5 mL, 0.52 mmol, 1 M in toluene). Catecholborane (1.1 mL, 10.41 mmol) was added dropwise whilst maintaining an internal temperature below −75° C. After the addition was completed, the reaction mixture was stirred overnight at −75° C. The reaction was monitored by TLC using ethyl acetate: hexane (3:7) as a mobile phase. The reaction mixture was quenched by slow addition of 4N HCl in 1,4-dioxane (0.4 mL) whilst maintaining the internal temperature below −75° C. The ice bath was removed and the reaction mixture was allowed to warm to room temperature. The reaction mixture was quenched into water and extracted with ethyl acetate and washed with water and brine. The organic phase was dried (MgSO₄) and the solvent removed under reduced pressure. The crude product was purified by column chromatography using ethyl acetate: hexane (3:7) to afford (S)-2,2,2-trifluoro-1-(4-fluorophenyl)ethanol as colourless liquid (0.97 g, 96%). Chiral HPLC: 92.7% (ee 85.4%)

### (S)-2,2,2-Trifluoro-1-(4-fluorophenyl)ethanol, triflate

Trifluoromethanesulfonic anhydride (0.78 mL, 4.63 mmol) was added dropwise to an ice-cooled solution of (S)-2,2,2-Trifluoro-1-(4-fluorophenyl)ethanol (0.5 g, 2.57 mmol) and 2,6-lutidine (0.60 mL, 5.15 mmol) in dichloromethane (5 mL). The mixture was stirred for 10 min under cooling and then poured into diethyl ether and washed rapidly with aqueous hydrochloric acid (pH=5) and brine. The solution was dried (MgSO₄) and the solvent removed under reduced pressure without heating. The crude triflate was obtained as unstable pink oil (1.7 g) which was used immediately in the next step.

### (R)-5-Methyl-8-[2,2,2-trifluoro-1-(4-fluorophenyl)ethyl]-7,8-dihydro-6H-pyrrolo[3,2e][1,2,4]triazolo[1,5-a]pyrimidine

5-Methyl-7,8-dihydro-6H-pyrrolo[3,2-e][1,2,4]triazolo[1,5-a]pyrimidine (0.3 gm, 1.72 mmol) was added to a stirred suspension of sodium hydride (60% dispersion in mineral oil, 0.07 gm, 0.67 mmol) in DMF (20 mL). After 30 min, the crude triflate (0.84 gm, 2.57 mmol) was added and the mixture was stirred at room temperature overnight. The reaction was monitored by TLC using dichloromethane:methanol (9:1) as a mobile phase. The mixture was diluted with ethyl acetate, washed with water and brine and dried (MgSO₄). The solvents were removed under reduced pressure. The crude product was purified by column chromatography using dichloromethane:methanol (9:1) to afford (R)-5-methyl-8-[2,2,2-trifluoro-1-(4-fluorophenyl)ethyl]-7,8-dihydro-6H-pyrrolo[3,2e][1,2,4]triazolo[1,5-a]pyrimidine as light brown solid. The compound was then submitted for chiral prep HPLC purification.
Column: CHIRALPAK AD 250×10 mm ID 10 micron
Flow rate: 12.0 ml/min
Wave length: 225 nm
Sample preparation: 8 mg/ml in 3-4 drops of MeOH: n-Hexane:Isopropyl alcohol (97:3)
Injection volume: 1.0 ml
Mobile phase (isocratic): n-Hexane:Isopropyl alcohol (97:3)
Run-time: 32 min
Yield of the pure enantiomer: 23 mg

¹H NMR (CDCl₃) δ 2.45 (3 H, s), 3.03-3.15 (1 H, m), 3.16-3.26 (1 H, m), 3.64 (1 H, m), 4.05 (1 H, m), 7.13 (2 H, t), 7.45-7.50 (2H, m), 7.62 (1 H, q) and 8.38 (1 H, s).

Mass: (ES+) 352 (M+H)⁺

Chiral Prep HPLC: 99.8% (ee 99.6%).

### Example 3

### (R)-5-Methyl-8-[2,2,2-trifluoro-1-(3,4-difluorophenyl)ethyl]-7,8-dihydro-6H-pyrrolo[3,2e][1,2,4]triazolo[1,5-a]pyrimidine [Compound 3]

This compound was prepared by the same method as used in Example 2 using (S)-2,2,2-trifluoro-1-(3,4-difluorophenyl)ethanol triflate.

¹H NMR (CDCl₃) δ 2.40 (3 H, s), 3.00-3.10 (1 H, m), 3.12-3.23 (1 H, m), 3.60 (1 H, m), 3.98 (1 H, m), 7.13-7.20 (2H, m), 7.28 (1H, m), 7.55 (1 H, q) and 8.30 (1 H, s).

Mass: (ES+) 370 (M+H)⁺

Chiral Prep HPLC: 99.4% (ee 98.8%).

### Example 4

### (S)-8-[1-(Phenyl)ethyl]-5-methyl-7,8-dihydro-6H-pyrrolo[3,2e][1,2,4]triazolo[1,5-a]pyrimidine [Compound 5]

Compound 5 is the (S)-enantiomer of RS-1178, previously discussed in Uryu et al (2002) Brain Research, 946(2), 298-306 and Uryu et al (2003) Biochem. Biophys. Res. Com., 303(1), 302-305. General synthetic routes to this compound are disclosed in Sato et al., J. Med. Chem. (1980), 23, 927-937. Compound 5 was prepared by the same method as used for the preparation of (S)-8-[1-(4-fluorophenyl)ethyl]-5-methyl-7,8-dihydro-6H-pyrrolo[3,2e][1,2,4]-triazolo[1,5-a]pyrimidine except that (S)-α-methylbenzylamine (ee>99.0%) was used in place of (S)-4-fluoro-α-methylbenzylamine.

¹H NMR (CDCl₃) δ 1.71 (3 H, d), 2.35 (3 H, s), 3.10 (2 H, m), 3.50 (1 H, m), 4.00 (1 H, m), 6.73 (1 H, q), 7.30-7.45 (5 H, m) and 8.48 (1 H, s).

MS (ES⁺): 280 (MH⁺, 100%).

### Example 5

### (R)-8-[1-(Phenyl)ethyl]-5-methyl-7,8-dihydro-6H-pyrrolo[3,2e][1,2,4]triazolo[1,5-a]pyrimidine [Compound 6]

Compound 6 is the (R)-enantiomer of RS-1178, previously discussed in Uryu et al (2002) Brain Research, 946(2), 298-306 and Uryu et al (2003) Biochem. Biophys. Res. Com., 303(1), 302-305. General synthetic routes to this compound are disclosed in Sato et al., J. Med. Chem. (1980), 23, 927-937. Compound 6 was prepared by the same method as used for the preparation of (S)-8-[1-(phenyl)ethyl]-5-methyl-7,8-dihydro-6H-pyrrolo[3,2e][1,2,4]-triazolo[1,5-a]pyrimidine except that (R)-α-methylbenzylamine (ee>99.0%) was used in place of (S)-α-methylbenzylamine.

¹H NMR (CDCl₃) δ 1.71 (3 H, d), 2.35 (3 H, s), 3.10 (2 H, m), 3.50 (1 H, m), 4.00 (1 H, m), 6.73 (1 H, q), 7.30-7.45 (5 H, m) and 8.48 (1 H, s).

MS (ES⁺): 280 (MH⁺, 100%).

### Example 6

### (S)-8-[1-(4-Fluorophenyl)ethyl]-5-methyl-7,8-dihydro-6H-pyrrolo [3,2e][1,2,4]triazolo[1,5-a]pyrimidine [Compound 7]

A mixture of intermediate 7-chloro-6-(2-chloroethyl)-5-methyl[1,2,4]triazolo[1,5-a]pyrimidine (390 mg, 1.7 mmol), (S)-4-fluoro-α-methylbenzylamine (378 mg, 2.7 mmol, ee 99%) and sodium carbonate (324 mg, 3.0 mmol) in ethanol (5 mL) was heated under reflux for 5 h. The mixture was cooled to room temperature, filtered, and the solvent removed from the filtrate. The crude product was triturated with diisopropylether to afford 8-[(S)-1-(4-fluorophenyl)ethyl]-5-methyl-7,8-dihydro-6H-pyrrolo[3,2e][1,2,4]triazolo[1,5-a]pyrimidine as a yellow solid (430 mg, 85%).

¹H NMR (CDCl₃) δ 1.71 (3 H, d), 2.39 (3 H, s), 3.06 (2 H, m), 3.44 (1 H, m), 3.84 (1 H, m), 6.85 (1 H, q), 7.03 (2 H, m), 7.34 (2 H, m) and 8.30 (1 H, s).

LCMS (ES⁺): 298 (MH⁺, 100%).

### Example 7

### (R)-5-Methyl-8-[2,2,2-trifluoro-1-phenylethyl]-7,8-dihydro-6H-pyrrolo[3,2e][1,2,4]-triazolo[1,5-a]pyrimidine [Compound 1, alternative method]

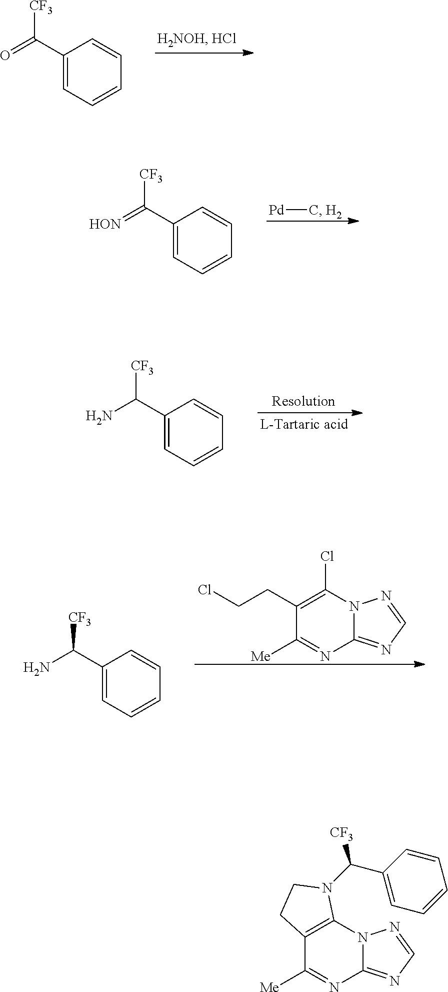

### 2′,2′,2′-Trifluoroacetophenone oxime

A mixture of 2′,2′,2′-trifluoroacetophenone (1500 g), hydroxylamine hydrochloride (1095 g), sodium acetate (1613 g), water (5.8 L) and ethanol (3 L) was heated to 75° C. and stirred for 24 h. The mixture was evaporated to about half of its original volume and allowed to stand overnight to crystallise. It was then filtered and washed with cold water followed by hexane. The crystalline solid was tray dried at 20° C. for two days. The resulting solid, 2′,2′,2′-trifluoroacetophenone oxime, (1660 g) was used directly in the next step.

### Racemic 2,2,2-Trifluoro-1-phenylethylamine

2′,2′,2′-Trifluoroacetophenone oxime (3320 g) was hydrogenated in eight equal parts. Each part (415 g) was dissolved in a mixture of tetrahydrofuran (2.1 L) and acetic acid (159 g) and hydrogenated at 50 psi and 60° C. in a Parr shaker using 5% palladium on carbon (30 g, Johnson Matthey type 87 L paste, 57% water) as catalyst. Once the reaction had set in at 60° C., the mixture exothermed to 85° C. The reaction maintained a temperature of 85° C. for 20-30 minutes before cooling (hydrogen uptake also complete). The mixture was hydrogenated for a further hour at 60° C. to ensure it was complete. It was then filtered through celite and evaporated to remove most of the tetrahydrofuran. All eight parts were combined before adding ice (4 kg) and water (4 L). The stirred mixture was adjusted to pH 14 with 32% sodium hydroxide solution (about 2.8 L required). The mixture was extracted with methyl tert-butyl ether (10 L, then 2×5 L). The combined extracts were dried over a mixture of anhydrous potassium carbonate and sodium sulphate and the solvent evaporated to give a light brown oil (2580 g, GC>99%). This material was used directly in the next step without distillation.

### R-(−)-2,2,2-Trifluoro-1-phenylethylamine

A mixture of racemic 2,2,2-trifluoro-1-phenylethylamine (2580 g), L-tartaric acid (2255 g) and isopropanol (45 L) was heated (in three equal parts using 20 L flange flasks) to form a solution at 70° C. The reaction mixtures were allowed to stand while slowly cooling overnight to room temperature. The resulting solid salt was filtered, washed with cold isopropanol (˜3 L) and combined. The mother liquor, mainly containing the (S)-isomer salt was kept separate. The damp solid salt was recrystallised from isopropanol (35 L) overnight. The resultant material salt was recrystallised three times from isopropanol (20 L for each recrystallisation). At this stage a small sample was worked-up to provide the amine free base. Chiral HPLC indicated it had 91% ee. This material was recrystallised twice from isopropanol (18 L for each recrystallisation). At this stage a small sample was worked-up to provide the amine free base. Chiral HPLC indicated it had 99.5% ee. This material was dried at 40° C. in a circulating air cabinet to give the tartrate salt (1476 g). This was added to ice (˜2 kg) and 32% sodium hydroxide solution (1138 ml) was added while stirring. The mixture was extracted with diethyl ether (3×2 L). The combined ether layers were dried over a mixture of anhydrous potassium carbonate and sodium sulphate and the solvent evaporated to give R-(−)-2,2,2-trifluoro-1-phenylethylamine as a colourless oil (732 g, 28%) (chiral HPLC indicated 99.2% ee) which crystallised on standing.

### 5-Methyl-8-(R-2,2,2-trifluoro-1-phenyl-ethyl)-7,8-dihydro-6H-pyrrolo[3,2-e][1,2,4]triazolo[1,5-a]pyrimidine

A mixture of 7-chloro-6-(2-chloroethyl)-5-methyl-[1,2,4]triazolo[1,5-a]pyrimidine (125 g) and R-(−)-2,2,2-trifluoro-1-phenylethylamine (105 g, 99.2% ee) was dissolved in N,N-diisopropylethylamine (3 L) and stirred at 125° C. for 18 h under a nitrogen atmosphere. A two phase mixture formed with an upper yellow/orange solution and a lower thick black oil. The hot N,N-diisopropylethylamine solution was decanted from the black oil and evaporated under vacuum to give an orange semi-solid. The N,N-diisopropylethylamine distillate was re-used to extract the black oil again by stirring at 125° C. for 30 minutes before decanting and evaporating again. The black oil extraction was repeated a total of three times. The combined orange semi-solid extracts were washed with hexane to give the crude product as a sticky solid.

A total of 125 g of crude 5-methyl-8-(R-2,2,2-trifluoro-1-phenyl-ethyl)-7,8-dihydro-6H-pyrrolo[3,2-e][1,2,4]triazolo[1,5-a]pyrimidine was prepared from 350 g of 7-chloro-6-(2-chloroethyl)-5-methyl-[1,2,4]triazolo[1,5-a]pyrimidine. The crude product was purified using silica gel flash chromatography (Biotage) eluting with 1% methanol in dichloromethane. Product containing fractions were evaporated to give a solid. This solid was dissolved in a mixture of dichloromethane (˜300 ml) and hexane (˜300 ml) and evaporated under vacuum until a solid had just started to form. Hexane (˜300 ml) was added and the mixture evaporated again under vacuum until a thick slurry formed. This material was filtered and washed with a cold hexane and vacuum dried at 50° C. overnight to give 5-methyl-8-(R-2,2,2-trifluoro-1-phenyl-ethyl)-7,8-dihydro-6H-pyrrolo[3,2-e][1,2,4]triazolo[1,5-a]pyrimidine as a yellow solid (67 g, 13%).

Found: C 57.43, H 4.21, N 20.87% C₁₆H₁₄F₃N₅ requires C 57.66, H 4.23, N 21.01%. HPLC analysis indicated that the chemical purity was 98.5% and chiral HPLC indicated >99.5% ee. Melting point: 153-155° C.

### Experimental Examples

### 1. Inhibition of β-amyloid induced activation

Mouse BALB/c monocyte macrophages, J774.2, {ECACC 85011428} were grown and sub-cultured in cell media (DMEM containing 10% FBS, 1% L-glutamine and 1% penicillin/streptomycin). The J774 cells were plated at 100,000 cells/well in 50 μl cell media on 96 well plates and placed in a 37° C., 5% CO₂ incubator overnight prior to experiments.

The addition of Aβ(1-42) and compounds to the J774 cells was performed using a Biotek precision 2000 liquid handling instrument. 3 μl of compound in DMSO ranging from 8 μM to 6 mM were pipetted into a “daughter plate” containing 294 μl of cell media and mixed thoroughly. 3 μl of Aβ(1-42) in DMSO at 4 mM was then added to the “daughter plate” and mixed thoroughly. 50 μl was then removed from the “daughter plate” and added to the plated J774 cells. The final concentrations in the wells containing 100 μl cell media were 20 μM Aβ(1-42), the compounds ranged from ˜40 nM to 30 μM in 1% DMSO and also in the presence of 50 U/ml Interferon gamma. The plates were incubated for 24 hours in a 37° C., 5% CO₂ incubator. After 24 hours incubation the media from the wells were collected and stored at −20° C. until required for testing.

The nitric oxide levels in the media were tested using the Griess assay (Promega G2930) using the manufacturer's instructions.

The TNF-alpha levels in the media were tested using a TNF-alpha ELISA (R&D Systems MTA00) or Meso Scale Discovery MS6000 Mouse Proinflammatory-7 kit, using the manufacturers instructions.

### 2. In Vitro ADME Data

### a) Cell Permeability of Compounds

These were measured using the accepted in vitro intestinal epithelial model using either the human colon adeno-carcinoma cell line Caco-2 or a sub-clone of this cell line, TC7. The apparent permeability coefficient (Pₐₚₚ) was determined in the apical-to-basolateral (A-B) and the B-A directions across cell monolayers cultured on 96-well polycarbonate membrane filters. Compounds were tested at 10 μM with a final DMSO concentration of 1%. The assay plate was incubated for 60 minutes at 37° C. with gentle shaking. Samples were taken at time zero from the donor side and at the end of the incubation from both donor and receiver sides. Samples were analysed by HPLC-MS/MS. The Pₐₚₚ value (expressed as 10⁻⁶ cm/sec) was then calculated based on the appearance rate of the compound in the receiver side (Gres, M. C. et al. (1998) Pharm. Res., 15, 726-733).

### b) Metabolism of Compounds

These were measured by incubating 1 μM test compound with pooled liver microsomes (0.3 mg/ml) protein for 60 minutes at 37° C. Samples were analysed by HPLC-MS/MS (Kuhnz, W. and Gieschen, H. (1998) Drug Metab. Dispos., 26, 1120-1127).

### c) Inhibition of Cytochrome P540s

Tests were carried out using human recombinant enzymes CYP1A2, CYP2C9, CYP2C19, CYP2D6 and CYP3A4 and P450-Glo™ screening system (Promega Corporation). Briefly, using a white opaque 96-well plate, one-fourth of the final reaction volume of a 4×cytochrome P450 reaction mixture containing the requisite luminogenic substrate was combined with an equal volume of the test compound (0.03-30 μM) or known inhibitor control at a 4×concentration to give one-half of the final reaction volume. A 10-minute pre-incubation was performed. One-half the final volume of the 2×NADPH regeneration system was then added to initiate the CYP reactions, and bring all components to their 1×target concentrations. The plate is incubated at 37° C. for 30 minutes. The luciferin detection reagent was added which stops the CYP reaction and initiates luminescence. The signal was allowed to stabilize for 20 minutes at room temperature and then the luminescence was read. The net CYP-dependent luminescence was calculated by subtracting the average luminescence of the control reactions from the CYP-containing reactions. Changes from the average net signal of untreated CYP reactions for reactions with a test compound reflect the inhibition of CYP activity by this compound.

TABLE 1
Identification of the active enantiomer

| Compound | Compound | IC₅₀ (μM)* |
|---|---|---|
| 4 | 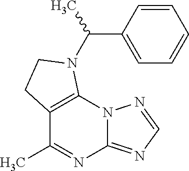 | 3 |
| 5 | 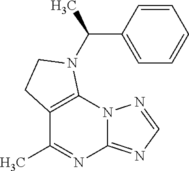 | 1.5 |
| 6 | 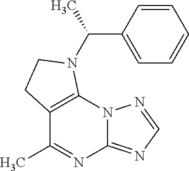 | 30 |

*IC₅₀ measured by inhibition of Nitric Oxide release (micromolar)

TABLE 2

| Compound | IC₅₀ (μM) TNF-α | IC₅₀ (μM) NO |
|---|---|---|
| 1 | 1.44* | 1.1 |
| 2 | 0.91*, 3.2** | 0.8 |
| 3 (assayed as the | N.D. | 1.8 |
| racemate) | | |
| 4 | 5.5** | 3 |
| 5 | 2.3** | 1.5 |

N.D. = Not determined
*Level of TNF for Aβ(1-42) alone: 9699 pg/mL
**Level of TNF for Aβ(1-42) alone: 13184 pg/mL

TABLE 3
In Vitro ADME data

| | | | Micro- | | | | | | |
|---|---|---|---|---|---|---|---|---|---|
| | Cell Perm (Pₐₚₚ | | somes t_{1/2} | | CYP450 Inhibition | | | | |
| | 10⁻⁶ cm s⁻¹) | | (mins) | | (IC₅₀ microM) | | | | |
| Compound | A-B | A-B/B-A | Human | Rat | 1A2 | 2C9 | 2C19 | 2D6 | 3A4 |
| 1 | 35 | 0.55 | >60 | 35 | >20 | 14 | >10 | >20 | 8 |
| 2 | 72 | 0.37 | >60 | 60 | >20 | >20 | >20 | >20 | 19 |
| 5 | 35 | 0.85 | >60 | 15 | >100 | >100 | 13 | 21 | 0.53 |
| 7 | 33 | 0.85 | >60 | 15 | 99 | 27 | 7 | 15 | 0.83 |

The compounds therefore have improved ADME characteristics compared to other compounds of the class and particularly a reduced ability to inhibit CYP450 such as 2D6 and 3A4.

1. A compound of the formula (I) or the pharmaceutically acceptable salt thereof 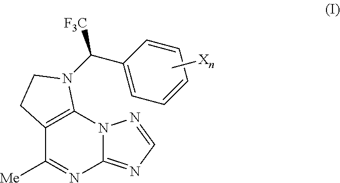
wherein
X is halogen, independently selected from fluorine and chlorine; and n is 0, 1 or 2.

2. A compound or the pharmaceutically acceptable salt thereof according to claim 1 wherein X is fluorine.

3. A compound or the pharmaceutically acceptable salt thereof according to claim 1 in which n is 1 or 2.

4. A compound or the pharmaceutically acceptable salt thereof according to claim 1 in which n is 0.

5. A compound or the pharmaceutically acceptable salt thereof according to claims 1 in which X is in the 3-position, the 4-position, or is in the 3- and 4-positions;

6. A compound or the pharmaceutically acceptable salt thereof according to claim 5 wherein X is in the 4-position, or is in the 3- and 4-positions;

7. A compound or the pharmaceutically acceptable salt thereof according to claim 5 wherein X is in the 4-position.

8. A compound according to any preceding claim selected from the group consisting of: 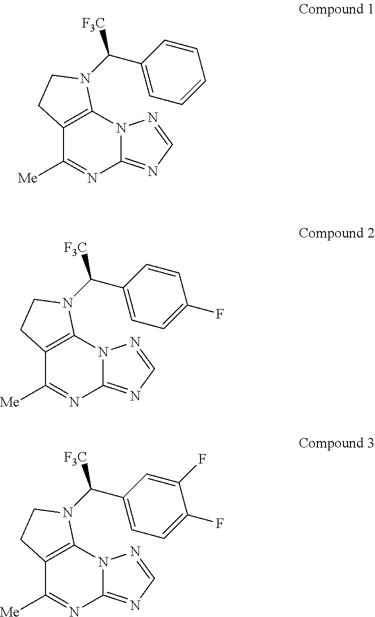
Or the pharmaceutically acceptable salt thereof.

9. A pharmaceutical composition comprising a compound of the formula (I) or a pharmaceutically acceptable salt thereof in combination with a pharmaceutically acceptable carrier or diluent.

10. A pharmaceutical composition as claimed in claim 9 which further comprises one or more compounds selected from anti-inflammatory compounds (eg NSAIDS), compounds suitable for the treatment of Alzheimer's disease (eg beta-amyloid) aggregation inhibitors, gamma-secretase inhibitors, gamma-secretase modulators or beta-secretase inhibitors) or compounds for the treatment of Parkinson's disease.

11. A composition comprising a compound according to claim 1, together with one or more additional therapeutic compounds for simultaneous, sequential or separate use.

12. A composition as claimed in claim 11 wherein the additional therapeutic compound is selected from anti-inflammatory compounds (e.g. NSAIDS), compounds suitable for the treatment of Alzheimer's disease (e.g. beta-amyloid aggregation inhibitors, gamma-secretase inhibitors, gamma-secretase modulators or beta-secretase inhibitors) or compounds for the treatment of Parkinson's disease.

13. A method of using in therapy comprising administering a compound of the formula (I) or a pharmaceutically acceptable salt thereof according to claim 1.

14. A method of treatment of a disease involving the activation of microglia, comprising administering to a patient to a patient in need thereof, a therapeutically effective amount of a compound of the formula (I) or a pharmaceutically acceptable salt thereof according to claim 1.

15. A method of treatment according to claim 14, wherein the disease involves the activation of microglia is Alzheimer's disease.

16. A method for treatment of a disease involving the activation of microglia comprising administering a medicament comprising a compound of the formula (I) or a pharmaceutically acceptable salt thereof according to claim 1.

17. The method according to claim 16 wherein the disease involving the activation of microglia is Alzheimer's disease.

18. A compound of the formula (I) or a pharmaceutically acceptable salt thereof according to claim 1 for the treatment of diseases involving the activation of microglia.

19. A compound of the formula (I) or a pharmaceutically acceptable salt thereof according to claim 1 for the treatment of Alzheimer's disease.
